(19) United States
(12) Patent Application Publication
(10) Pub. No.: US 2022/0298540 A1
(43) Pub. Date: Sep. 22, 2022

(54) METHOD AND REAGENTS FOR DETECTING LUCIFERASE ACTIVITY

(71) Applicant: OBSCHESATVO S OGRANICHENNOY OTVETSTVENNOSTYU "PLANTA", Moscow (RU)

(72) Inventor: Il'ya Viktorovich YAMPOL'SKIY, Moscow (RU)

(21) Appl. No.: 17/633,843

(22) PCT Filed: Apr. 14, 2020
(86) PCT No.: PCT/RU2020/050074

§ 371 Date: Feb. 8, 2022

(30) Foreign Application Priority Data
Aug. 8, 2019 (RU) 2019125190

(51) Int. Cl. C12Q 1/66; C12N 9/02; C07D 495/04

(52) U.S. Cl. CPC C12Q 1/66; C12N 9/0004; C12Y 113/12; C07D 495/04

(57) ABSTRACT

The bioluminescent system components are commonly used reagents for a variety of analyzes, including diagnostic systems, quality control systems, drug testing systems, etc. This group of inventions discloses the components of the bioluminescent system Odontosyllis undecimdonta worm, specifically luciferin and pre-luciferin. Besides, this group of inventions discloses a method for detecting luciferase in biological samples using luciferin and pre-luciferin of Odontosyllis undecimdonta worm, and also a method for detecting bioluminescence in a biological sample.

### FIELD OF THE INVENTION

This present invention relates to biology, chemistry and biotechnology, specifically the bioluminescent system of Odontosyllis undecimdonta worm.

### BACKGROUND OF THE INVENTION

Bioluminescence—emission of light by living organisms during biochemical reaction wherein chemical energy is converted into light energy. Bioluminescence capability is defined by availability of a specific luciferase protein or photoprotein. Luciferases—enzymes, which catalyze oxidation of low-molecular compounds—luciferins, transforming them into oxyluciferins. Oxidation is accompanied by light emission and oxyluciferin release. There are several disclosed bioluminescent systems.

For example, a number of sea coelenterates is characterized by the systems comprising proteins of aequorin family (Prasher, et al., Biochem. 1987, 26:1326-1332; Tsuji et al., Photochem Photobiol, 1995 62(4):657-661). Aequorin family also includes obelin, halistaurin=mitrocomin, phiallidin=clytin, etc. These are photoproteins containing a covalently bound luciferin, that in the presence of Ca²⁺ ions, is subjected to chemical transformation forming a product in excited electronic state.

Marine polychaetes Odontosyllis undecimdonta, widely known as “fireworms”, emit bright blue-green luminescence. Odontosyllis bioluminescence is the luciferin-luciferases system, luciferase protein is known from Darrin T. Schultz et al, Biochemical and Biophysical Research Communications, V. 502 (3), 2018, P. 318-323, however, the luciferin structure has not been known.

The components of bioluminescent systems (luciferases, photoproteins, luciferins, etc.)—are commonly used reagents for a variety of analytical tests, including diagnostic systems, quality control systems, etc. For example, proteins of aequorin family are widely used for researches of Ca²⁺ release and binding in biological systems, for example, during muscular contraction. Use of bioluminescent systems is described in details, for example, in Cormier, M. L. et al., Photochem. & Photobiol. 49/4, 509-512 (1989), Smith, D. F. et al. in “Bioluminescence and Chemiluminescence: Current Status (P. Stanley & L. Krick, eds.), John Wiley and Sons, Chichester, U.K. 1991, 529-532.

In spite of a variety of bioluminescent systems used today there remains a need for expanding the range of luciferin-luciferase pairs with new properties. Interpretation of bioluminescent system new components enables to extend the spectrum of analyses and applications available for use.

### DISCLOSURE OF THE INVENTION

The object of this invention is identification of components of the bioluminescent system of Odontosyllis undecimdonta worm, specifically identification of luciferin and pre-luciferin molecule, and also development of a method for detecting luciferase in a biological sample using luciferin and pre-luciferin of Odontosyllis undecimdonta worm, and a method for detecting bioluminescence using luciferin and pre-luciferin of Odontosyllis undecimdonta worm.

The technical result is widening the range of technical means in the field of use of bioluminescent systems, and it is achieved due to identification of luciferin molecule of Odontosyllis undecimdonta worm, the oxidation of which is accompanied by light emission. The technical result is also achieved due to identification of pre-luciferin molecule of Odontosyllis undecimdonta worm. The said components of Odontosyllis undecimdonta worm bioluminescent system are promising for use as reagents for a variety of analyzes, including diagnostic systems, quality control systems, drug testing systems, etc.

This invention discloses a worm luciferin molecule, namely 4-hydroxy-5-(sulfooxy)-7H-thieno[3,2-f]thiochromen-1,7,8-tricarboxylic acid, characterized by the following structural formula:

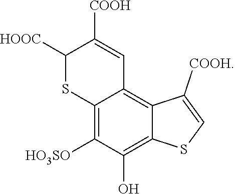

and/or its tautomer—4-hydroxy-5-(sulfooxy)-9H-thieno[3,2-f]thiochromen-1,7,8-tricarboxylic acid, characterized by the following structural formula:

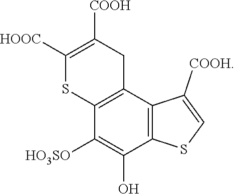

This invention also discloses use of the compound 4-hydroxy-5-(sulfooxy)-7H-thieno[3,2-f]thiochromen-1,7,8-tricarboxylic acid and/or its tautomer—4-hydroxy-5-(sulfooxy)-9H-thieno[3,2-f]thiochromen-1,7,8-tricarboxylic acid as a substrate for luciferase enzyme, oxidation of this molecule causes luminescence.

This invention also discloses a pre-luciferin molecule, namely the compound 7-(carboxycarbonyl)-4-hydroxy-5-(sulfooxy)-9H-thieno[3,2-f]thiochromene-1,8-dicarboxylic acid, characterized by the following structural formula:

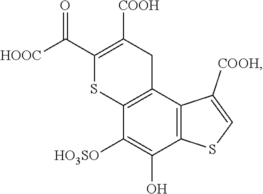

and/or its tautomer 7-(carboxycarbonyl)-4-hydroxy-5-(sulfooxy)-7H-thieno[3,2-f]thiochromene-1,8-dicarboxylic acid, characterized by the following structural formula:

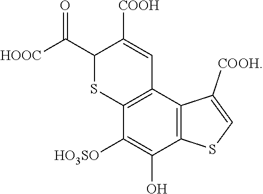

This invention also discloses use of the compound 7-(carboxycarbonyl)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid as a precursor of luciferin (pre-luciferin).

This invention also includes a kit for detecting luciferase in a biological sample comprising luciferin and/or pre-luciferin of the invention.

In particular embodiments the kit additionally includes a buffer, detergent, reducing agent and/or glycerol.

In particular embodiments said kit components are in permissible amounts.

This invention also includes a bioluminescent composition, comprising luciferase, and at least one compound of this invention. In particular embodiments a luciferase is recombinant. In particular embodiments a luciferase is Odontosyllis undecimdonta worm luciferase.

Besides, this invention also discloses a method for detecting luciferase in a biological sample comprising luciferase, and at least one compound of the invention. In particular embodiments a luciferase is Odontosyllis undecimdonta worm luciferase. In particular embodiments a luciferase is a recombinant luciferase. In particular embodiments a biological sample is a tissue and/or cell. In particular embodiments a biological sample is characterized by pH within the range from 7 to 8.

In particular embodiments a method of detecting luciferase includes the following steps:
a) adding the compound of the invention to a biological sample to obtain a reaction mixture;
b) incubating the reaction mixture in conditions suitable for bioluminescence;
c) detecting bioluminescence in the reaction mixture.

In particular embodiments the compound (luciferin and/or pre-luciferin) concentration is 0.03-300 μM.

Besides, this invention also discloses a method for detecting bioluminescence in a biological sample comprising luciferase, and at least one compound of the invention. In particular embodiments a luciferase is a recombinant luciferase. In particular embodiments a luciferase is Odontosyllis undecimdonta worm luciferase. In particular embodiments a biological sample is a tissue and/or cell. In particular embodiments a biological sample is characterized by pH within the range from 7 to 8.

In particular embodiments a method of detecting bioluminescence includes the following steps:
a) expressing luciferase gene in a biological sample;
b) adding the compound of the invention to the biological sample;
c) detecting bioluminescence.

In particular embodiments the compound of the invention (luciferin and/or pre-luciferin) concentration is 0.03-300 μM.

This invention also discloses reagents and reagent kits for implementation of this invention methods.

This invention also includes obtaining the compounds of the invention.

Detailed specification of the invention summarized above is given below for complete disclosure of the above listed characteristics in the form of references to the embodiments, some of which are illustrated in additional figures. It is worth noting that the attached figures illustrate typical embodiments only, and, therefore, shall not be interpreted as limiting the scope of the invention, which could concede other embodiments equally effective.

### DETAILED DISCLOSURE OF THE INVENTION

### BRIEF DESCRIPTION OF THE FIGURES

FIG. 1. Result of measuring Odontosyllis undecimdonta luciferine bioluminescence over time.

FIG. 2. UV-vis spectrum of Odontosyllis undecimdonta pre-luciferin.

FIG. 3. UV-vis spectrum of Odontosyllis undecimdonta luciferin.

FIG. 4. Chromatographic profile of lyophilized Odontosyllis undecimdonta worm water extract based on the results of anion-exchange chromatography in DEAE Sepharose HiTrap Fast Flow column. Solid line—absorption signal at wave length of 280 nm. Odontosyllis luciferase and luciferin activity profiles in relative light units (RLU).

FIG. 5. Components of Odontosyllis undecimdonta polychaete worm bioluminescent system:
A) NMR tube with purified oxyluciferin, visible light;
B) UV-initiated oxyluciferin fluorescence.

FIG. 6. Reverse phase chromatographic profile of luciferin-containing fraction obtained from anion-exchange chromatography in 5 μm TSK ODS 120T 5 capillary column (monitored at two different wave length: 300 nm and 410 nm). Odontosyllis luciferin and oxyluciferin peaks respectively.

FIG. 7. Numeration of heavy atoms:
A) luciferin;
B) oxyluciferin;
C) pre-luciferin.

FIG. 8. Luciferin high resolution mass spectrum demonstrating the similarity with fragmentation patterns of Odontosyllis luciferin (noise peak is denoted by asterisk).

FIG. 9. Reverse phase chromatographic profile of luciferin-containing fraction based on the results of anion-exchange chromatography of lyophilized Odontosyllis undecimdonta worm water extract on YMC-Triart C18 column (3 μm, 12 nm, 75×4.6 mm) (control at Aabs =254 nm). Odontosyllis luciferin and pre-luciferin peaks are marked respectively.

FIG. 10. Pre-luciferin high resolution mass spectrum demonstrating the similarity with fragmentation patterns of Odontosyllis luciferin (noise peak is denoted by asterisk).

FIG. 11. Typical result of luciferase detection in biological samples.

FIG. 12. Typical result of bioluminescence detection in biological samples.

FIG. 13. Typical result of measuring Odontosyllis undecimdonta pre-luciferine bioluminescence over time.

### TERMS AND DEFINITIONS

Different terms referred to the subject matters of this invention are used above and also in the invention specification and claims. Unless otherwise specified, all technical and scientific terms used in this use have the same meaning, which is understood by those skilled in the art. References to the techniques used at describing this invention refer to the well-known methods, including changes of these methods and their substitution by equivalent methods known to those skilled in the art.

The terms “comprises” and “comprising” in the specification of this invention are interpreted as “comprises, but not limited to”. The said terms are not intended to be interpreted as “consists only of”.

The term “bioluminescence” or “luminescence” herein means a process of light emission as result of reaction between an enzyme and a substrate generating light.

The term “luciferin precursor (pre-luciferin)” herein means a compound able to transform to luciferin spontaneously or being induced by enzymes.

The term “luciferin” herein means a compound being a substrate for luciferase enzymes.

As it is used herein, the term “luciferase” means a protein, which is able to oxidize luciferin, where oxidation reaction is accompanied by light emission (luminescence), and oxidized luciferin is released.

“Luciferase reaction mixture” comprises luciferase enzyme and materials enabling luciferase enzyme to generate light signal. The required materials and specific concentrations and/or amounts of the materials required for luminescent signal generation vary depending on the luciferase enzyme used, and also on the type of the luciferase-based assay. Normally, for Odontosyllis undecimdonta worm luciferase these materials can include a buffer maintaining proper pH for the reaction, enzyme of Odontosyllis undecimdonta worm luciferase and luciferin. Other materials may often be added to the solution, including: bovine serum albumin (BSA) to maintain luciferase activity, reducing agents, detergents, salts, glycerol, amino acids, for example, D-cysteine, etc. A typical luciferase reaction mixture may comprise Odontosyllis undecimdonta worm luciferase, 50 mM sodium phosphate buffer pH 7.4, 5% glycerol.

“Luciferase detection mixture” comprises materials enabling to detect luciferase enzyme. The required materials and specific concentrations and/or amounts of the materials required for luminescent signal generation vary depending on the luciferase enzyme used, and also on the type of the luciferase-based assay. Generally, these materials for Odontosyllis undecimdonta worm luciferase may include: reducing agents, detergents, salts, glycerol, amino acids, luciferase substrate—luciferin. Other materials may often be added to the solution, including: bovine serum albumin (BSA) to maintain luciferase activity, reducing agents, detergents, salts, glycerol, amino acids, for example, D-cysteine, etc. A typical luciferase detection mixture may comprise a luciferase substrate — Odontosyllis undecimdonta worm luciferine, 50 mM sodium phosphate buffer pH 7.4

As used herein, the term “isolated” means a molecule or cell, which is in the environment different from the environment wherein the molecule or cell naturally occurs. For example, the said components can be in a substantially purified form. A substantially purified form means that proteins are at least about 20% pure, often at least 30% pure, normally 50% pure, or at least 90% pure.

Any conventional protein purification techniques described, for example, in Guide to Protein Purification, (Deuthser ed.) could be used for protein isolation. (Academic Press, 1990). For example, a lysate or cold extract could be prepared from the initial source and purified using HPLC, size-exclusion chromatography, gel electrophoresis, affinity chromatography, etc. Protein formulations can be tested for active luciferase or a luciferase-luciferin complex using the present invention methods.

As used herein, the term “mutant” or “derivative” refers to a protein (specifically to luciferase) disclosed in this invention, wherein one or more amino acids have been added and/or substituted and/or removed (deleted) and/or inserted at the N-terminus and/or C-terminus, and/or within the native amino acid sequences of proteins of this invention. As used herein, the term “mutant” refers to a nucleic acid molecule that encodes a mutant protein. Besides, the term “mutant” herein refers to any variant that is shorter or longer than a protein or nucleic acid.

Modifications and also additions or deletions could be introduced by any method known in the art (see, for example, Gustin et al., Biotechniques (1992) 14: 22; Barany, Gene (1985) 37: 11-123; and Colicelli et al., Mol. Gen. Genet. (1985) 199:537-539), Sambrook et al., Molecular Cloning: A Laboratory Manual, (1989), CSH Press, pp. 15.3-15.108), including misdirected PCR, shuffling, site-directed mutagenesis using oligonucleotides, mutagenesis using PCR based on paired molecules, in vivo mutagenesis, cassette mutagenesis, recursive matched mutagenesis, exponential matched mutagenesis, site-directed mutagenesis, random mutagenesis, gene reassembly, gene site saturated mutagenesis (GSSM), ligation synthesis reassembly (SLR), or a combination thereof. The said modifications, additions or deletions can also be introduced by a method including recombination, recursive sequence recombination, DNA mutagenesis by phosphothioate modification, mutagenesis based on the inclusion of a template comprising uracil, mutagenesis based on a duplex comprising gaps, repair mutagenesis with point misparings, mutagenesis using a repair-deficient host strain, chemical mutagenesis, radiogenic mutagenesis, deletion mutagenesis, mutagenesis using selectional restriction, mutagenesis using purification restriction, artificial gene synthesis, matched mutagenesis, creation of a chimeric nucleic acid multimer, or a combination thereof.

As used herein, the term “functional” means that a nucleotide or amino acid sequence can function for a specified test or task. The term “functional”, used to describe luciferases, means that a protein is able to produce a luminescent luciferin oxidation reaction.

### Biological Samples

Implementation of the methods of this invention ensures luminescence of the reaction mixture comprising a biological sample, if the said sample comprises luciferase used as a substrate (i.e. luciferin) 4-hydroxy-5-(sulfooxy)-7H-thieno[3,2-f]thiochromene-1,7,8-tricarboxylic acid and/or its tautomer 4-hydroxy-5-(sulfooxy)-9H-thieno[3,2-f]thiochromene-1,7,8-tricarboxylic acid. For example, such luciferase is contained in the bioluminescent marine polychaetes Odontosyllis undecimdonta.

Biological samples could be obtained using various techniques known in biology, and include tissue and cell samples, extracts, homogenates, protein mixtures of various purification degree, etc. For example, biological samples could be obtained from the marine polychaetes Odontosyllis undecimdonta.

Biological samples may also comprise isolated components (luciferase or luciferase and luciferin or pre-luciferin) of Odontosyllis undecimdonta polychaete bioluminescent systems.

Biological samples can also express recombinant luciferase or functional mutants thereof. The nucleic acid sequences for expression of the said proteins could be obtained from natural sources (for example, from Odontosyllis undecimdonta polychaetes) or be synthesized. At present, there is a variety of methods for cloning genes encoding proteins of known activity. Such methods are partially described in Maniatis, T., et al. (Molecular Cloning—A Laboratory Manual Cold Spring Harbor Laboratory, Cold Spring Harbor, N.Y. 1982) and Newman and Campagnoni (Neuromethods, v. 16, 1990, pp 13-48). For example, an expression library can be prepared in suitable host cells and tested for luciferase activity. Alternatively, protein could be isolated from a cold extract, its partial amino acid sequence determined and the corresponding cDNA cloned from a cDNA sample from Odontosyllis undecimdonta polychaetes. The nucleic acid sequences should be inserted into an expression cassette. The expression cassette may exist as an extrachromosomal element or it may be incorporated into a cell genome by introducing the said expression cassette into the cell. In the expression cassette, a protein encoding nucleic acid is operably linked to a regulatory sequence that may include promoters, enhancers, terminators, operators, repressors, and inducers. Once the expression cassette is inserted into a cell, a functional protein can be generated therein Expression systems include, for example, bacterial systems, yeast cells, insects, fish, amphibians, or mammalian cells. Methods for producing expression cassettes or systems for expressing a desired product are known to those skilled in the art. Cell lines that stably express luciferase could be selected by methods known in the art (for example, co-transfection with a selectable marker, such as dhfr, gpt, neomycin, hygromycin, which makes it possible to identify and isolate transfected cells that comprise a gene included into the genome). The above expression systems can be used in prokaryotic or eukaryotic hosts. Such host cells as E. coli, B. subtilis, S. cerevisiae, insect cells in combination with baculovirus vectors, or cells of a higher organism such as vertebrates, for example, COS 7 cells, HEK 293, CHO, Xenopus oocytes, etc. can be used for producing protein.

Functional mutants of natural proteins can also be expressed. As used in this specification, the term “functional” in relation to luciferase means that the said protein is able to use 4-hydroxy-5-(sulfooxy)-7H-thieno[3,2-f]thiochromene-1,7,8-tricarboxylic acid and/or its tautomer 4-hydroxy-5-(sulfooxy)-9H-thieno[3,2-f]thiochromene-1,7,8-tricarboxylic acid as luciferin.

The reference to a nucleotide sequence “encoding” a polypeptide means that this polypeptide is produced from the nucleotide sequence during mRNA translation and transcription. In this case, both the coding strand, identical to mRNA and usually used in the sequence listing, and the complementary strand, which is used as a transcription template, can be specified. As it is obvious to any person skilled in the art, the term also includes any degenerate nucleotide sequences encoding the same amino acid sequence. Nucleotide sequences encoding a polypeptide include sequences comprising introns.

### Reagents for Detecting Luciferase Activity

The methods of this invention are based on the use of 4-hydroxy-5-(sulfooxy)-7H-thieno[3,2-f]thiochromene-1,7,8-tricarboxylic acid and/or its tautomer 4-hydroxy-5-(sulfooxy)-9H-thieno[3,2-f]thiochromene-1,7,8-tricarboxylic acid for detecting luciferase activity in biological samples.

As used herein, 4-hydroxy-5-(sulfooxy)-7H-thieno[3,2-f]thiochromene-1,7,8-tricarboxylic acid is a compound having the following structural formula:

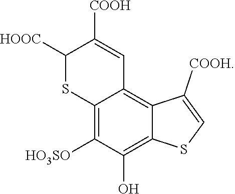

As used herein, the tautomer of the above compound is 4-hydroxy-5-(sulfooxy)-9H-thieno[3,2-f]thiochromene-1,7,8-tricarboxylic acid having the following structural formula:

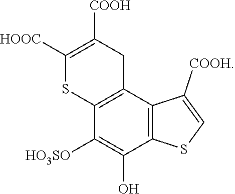

This new luciferin isolated by the present inventors has a unique structure that distinguishes it from all previously described natural luciferins. Thus, the basis of the Odontosyllis worm luciferin molecule is a condensed heterocycle—thieno[3,2-f]thiochromene consisting of two six-membered and one five-membered rings. Such complex condensed tricyclic structures have not previously been found as bioluminescent reaction substrates. It is also worth noting that thiopyran is named in the composition of natural bioluminescent substrates for the first time. Odontosyllis worm luciferin is a strongly polar molecule and represents a strong acid, since this molecule comprises three carboxyl substituents and also carries a sulfonic acid residue. Freeze-dried substrate is stored at −20° C. without decrease in activity for at least 30 days, more often at least 60 days, normally at least a year.

The present inventors have developed a pathway for synthesis of Odontosyllis worm luciferin (scheme 1). The recommended scheme of synthesis of the said compound includes 9 steps and uses 2,3-dimethoxyphenylthiol as a starting substance. Treatment of the starting dimethoxyphenylthiol with methyl propiolate in the presence of azoisobutyronitrile leads to formation of a bicyclic condensed system methyl 6,7-dimethoxybenzo[b]thiophene-3-carboxylate (1). Subsequent bicycle bromination and cross-coupling of halide 2 with ethyl acrylate by Suzuki reaction leads to the product 3 with high yield. Oxidation of the compound 3 when exposed to K₂OsO₄ and NaIO₄ gives methyl 4-formyl-6,7-dimethoxybenzo[b]thiophene-3-carboxylate (4). Removal of protective methyl groups and further oxidation of the product 5 with cerium ammonium nitrate results in the product 6 -methyl 4-formyl-6,7-dioxo-6,7-dihydrobenzo[b]thiophene-3-carboxylate. Wittig reaction between the compound 6 and dimethyl 2-(dimethoxyphosphoryl)-3-mercaptosuccinate under alkaline conditions leads to formation of the tricyclic condensed structure 7, subsequent esterification of which with pyridinesulfonic acid and removal of protective methyl groups leads to the target product 9—Odontosyllis worm luciferin.

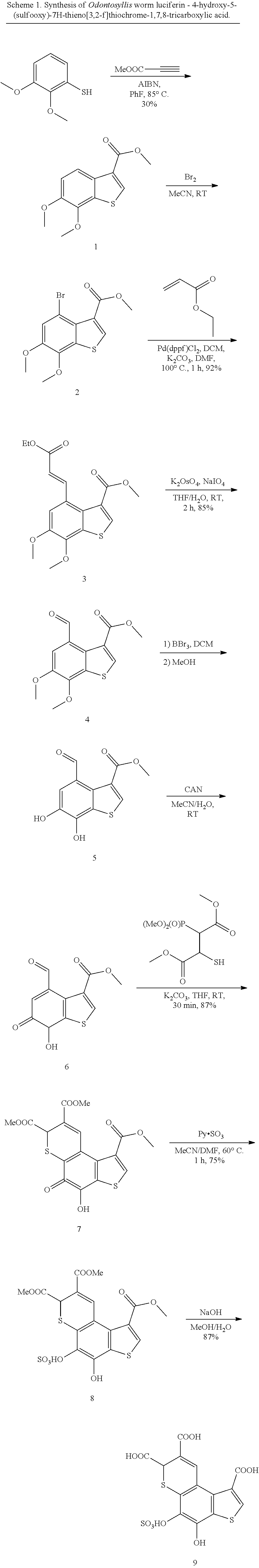

As used herein, 7-(carboxycarbonyl)-4-hydroxy-5-(sulfooxy)-9H-thieno[3,2-f]thiochromene-1,8-dicarboxylic acid is a compound characterized by the following structural formula:

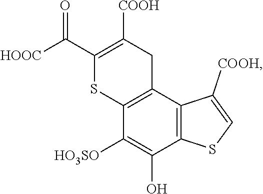

and/or its tautomer 7-(carboxycarbonyl)-4-hydroxy-5-(sulfooxy)-7H-thieno[3,2-f]thiochromene-1,8-dicarboxylic acid, characterized by the following structural formula:

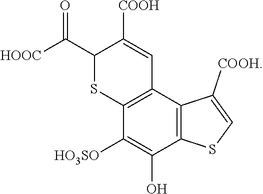

This new pre-luciferin isolated by the present inventors has a unique structure that distinguishes it from all previously described natural pre-luciferins.

### Composition of Conditions for Developing A Bioluminescent Signal

Formation of bioluminescence depends on the amount and preservation of luciferase in biological samples. It should also be noted the effect of light emission, especially the UV part of the spectrum, on luciferin, leading to its degradation and loss of functionality, that was confirmed experimentally.

Signal formation is influenced by the reaction mixture pH. Formation of a bioluminescent signal occurs in the pH range from 6.0 to 9.8, normally in the pH range from 6.5 to 9.0, predominantly in the range from 7.0 to 8.0. Any standard buffer solutions for a given pH range can be used to ensure pH, including phosphate buffer, HEPES, Tris-HCl. In preferred embodiments, the buffer solution molarity does not exceed 2, for example, does not exceed 1, more often in the range from 0.05 to 0.4, normally from 0.1 to 0.2.

Reaction mixtures for the needs of the present invention may also comprise the components stabilizing and protecting the bioluminescent system enzymes from the inhibitory effect of trace amounts of heavy metal ions and from degradation by proteases.

For example, the reaction mixture may comprise DTT at a concentration of max. 20 mM, more often at a concentration of 0.1 to 8 mM, predominantly at a concentration of 0.1 to 4 mM.

The reaction mixture may also comprise beta-mercaptoethanol and/or EDTA at a final concentration of 0 to 5 mM.

For example, the reaction mixture may comprise 0.1-2 mM DTT and 0.1-1 mM EDTA.

Also, the reaction mixture may comprise protease inhibitors, for example, phenylacetic acid or oxalic acid in standard concentrations.

For the needs of the present invention, 4-hydroxy-5-(sulfooxy)-7H-thieno[3,2-f]thiochromene-1,7,8-tricarboxylic acid and/or its tautomer 4-hydroxy-5-(sulfooxy)-9H-thieno[3,2-f]thiochromene-1,7,8-tricarboxylic acid is added to the biological sample to a final concentration of 0.03-300 μM, more often 1-5 μM.

For the needs of the present invention, 7-(carboxycarbonyl)-4-hydroxy-5-(sulfooxy)-9H-thieno[3,2-f]thiochromene-1,8-dicarboxylic acid and/or its tautomer 7-(carboxycarbonyI)-4-hydroxy-5-(sulfooxy)-7H-thieno[3,2-f]thiochromene-1,8-dicarboxylic acid is added to the biological sample to a final concentration of 0.03-300 μM, more often 1-5 μM.

In some embodiments a mixture of reagents is added to the sample, including a buffer solution, components stabilizing and protecting the bioluminescent system enzymes from the inhibitory effect of trace amounts of heavy metal ions and from degradation by proteases. In other embodiments, a buffer solution, components stabilizing and protecting the bioluminescent system enzymes from the inhibitory effect of trace amounts of heavy metal ions and from degradation by proteases are first added to the biological sample, and then a solution of 4-hydroxy-5-(sulfooxy)-7H-thieno[3,2-f]thiochromene-1,7,8-tricarboxylic acid and/or its tautomer 7-(carboxycarbonyI)-4-hydroxy-5-(sulfooxy)-7H-thieno[3,2-f]thiochromene-1,8-dicarboxylic acid or a solution of 7-(carboxycarbonyl)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid.

Depending on the solvent used for preparation of the luciferin solution or pre-luciferin solution, the reaction mixture may comprise small amounts of used solvents

Also, the reaction mixture may comprise detergents, such as Triton X100 or nonylphenoxypolyethoxyethanol. In some embodiments, the detergent concentration in the reaction mixture does not exceed 0.2%, more often does not exceed 0.1%, optimally does not exceed 0.06%.

Also, the reaction mixture may comprise bovine serum albumin (BSA) or other proteins at the concentration not exceeding 2%, more often not exceeding 1%, optimally not exceeding 0.5%. BSA is used when the biological sample concentration is extremely low, then BSA acts as a protein stabilizer.

Development of the bioluminescent signal occurs in a wide temperature range—from 4 to 40° C., optimally at 20-25° C.

Formation of a luminescent signal begins immediately after the reaction initiation with adding the above mentioned key reagents for detecting luciferase activity.

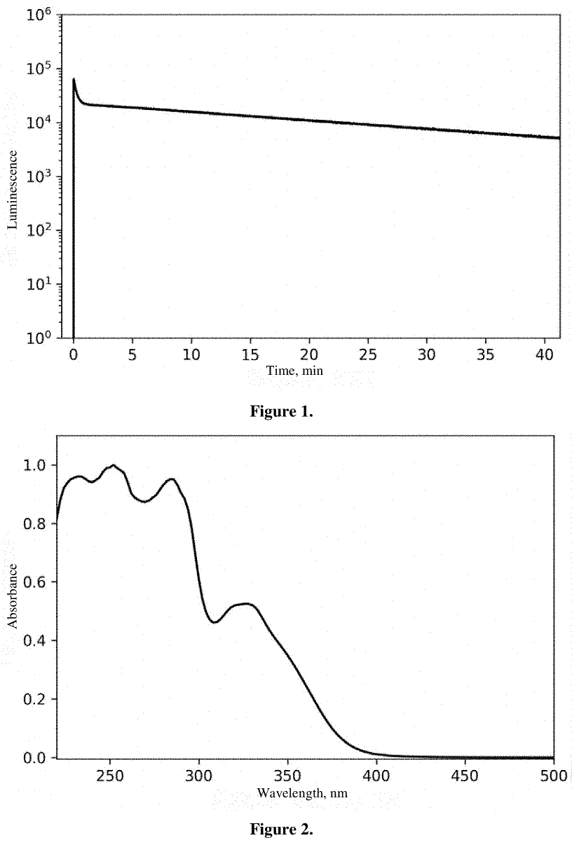

The maximum luminescence intensity is observed at the moment of the reaction initiation. Then there is a decay, the rate of which is determined by the activity of enzymes and initial concentrations of substrates. Under certain conditions (there are many substrates, enzyme activity is low, the reaction temperature is reduced), the reaction can be observed for 4 hours or more (FIG. 1).

### Bioluminescence Detection

The methods of this invention include detecting bioluminescence that occurs in a biological sample comprising luciferase at the appearance of luciferin therein.

Bioluminescence could be detected using the methods known to those skilled in the art, in particular by visual screening or using a luminometer, photometer, fluorimeter, digital camera, sensitive film. As a quantitative characteristic, the maximum luminescence intensity can be used, which is achieved 5-30 min after initiation of the bioluminescent reaction, or the rate of luminescence rise within the interval up to 30 min after initiation of the bioluminescent reaction, for example, within 5, 10, 20, 30, 60 sec. after the reaction initiation or longer.

In preferred embodiments, the measured luminescence is rather a persistent light emission than light flashes. In preferred embodiments, the luminescence intensity depends on the activity of the bioluminescent system enzymes present in the sample, substrate initial concentrations and the reaction mixture temperature and usually ranges from 10 kv/s to 10 million kv/s, more often 100-100,000 kv/s.

The reaction lasts at least 30 minutes after initiation, more often 30-60 minutes, sometimes (depending on the conditions) some hours and even days.

Light emitted at oxidation of 4-hydroxy-5-(sulfooxy)-7H-thieno[3,2-f]thiochromene-1,7,8-tricarboxylic acid and/or its tautomer 4-hydroxy-5-(sulfooxy)-9H-thieno[3,2-f]thiochromene-1,7,8-tricarboxylic acid is in the range from 450 to 600 nm, more often in the range from 475 to 550 nm, with emission maximum at 505-510 nm.

### Methods of Use

The methods and reagents of this invention can be used in a wide variety of in vivo and in vitro bioluminescence assays.

In particular, the methods and reagents of this invention can be used for detecting active components of the bioluminescent system of Odontosyllis undecimdonta polychaetes during purification thereof.

The methods and reagents of this invention can also be used for detecting functional analogues of the bioluminescent system enzymes of Odontosyllis undecimdonta polychaetes in biological samples.

The methods and reagents of this invention can also be used for detecting recombinant luciferase activity in host cells.

In some embodiments, a nucleic acid encoding a luciferase is to be obtained for implementation. The resulting nucleic acid should be inserted into an expression cassette providing for the temporary or permanent expression of this nucleic acid in host cells, for example, under promoters of interest to a researcher. An expression cassette may comprise elements ensuring targeted construct delivery to the cells or cell compartments of interest, or be contained in particles providing targeted delivery. After cell transfection with an expression cassette (for example, as part of an expression vector) and upon the expiry of time required for producing an expression product in the cells, the luciferase activity can be detected inside the cells or in the cell lysate.

### Kits

Kits for use in the above applications are also provided in accordance with this invention.

In some embodiments, the kits typically include 4-hydroxy-5-(sulfooxy)-7H-thieno[3,2-f]thiochromene-1,7,8-tricarboxylic acid and/or its tautomer 4-hydroxy-5-(sulfooxy)-9H-thieno[3,2-f]thiochromene-1,7,8-tricarboxylic acid, preferably with a buffer solution for dissolving the specified substrate and/or adding it to biological samples. 4-hydroxy-5-(sulfooxy)-7H-thieno[3,2-f]thiochromene-1,7,8-tricarboxylic acid and/or its tautomer may be dissolved in an appropriate storage medium, such as aqueous or buffer solution with detergent, normally, in an appropriate vessel. Alternatively, 4-hydroxy-5-(sulfooxy)-7H-thieno[3,2-f]thiochromene-1,7,8-tricarboxylic acid and/or its tautomer may be lyophilized in the kit.

In some embodiments, the kits typically include 7-(carboxycarbonyl)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid, preferably with a buffer solution for dissolving the specified compound and/or adding it to biological samples. 7-(carboxycarbonyl)-4-hydroxy-5-(sulfooxy)-9H-thieno[3,2-f]thiochromene-1,8-dicarboxylic acid and/or its tautomer 7-(carboxycarbonyI)-4-hydroxy-5-(sulfooxy)-7H-thieno[3,2-f]thiochromene-1,8-dicarboxylic acid may be dissolved in an appropriate storage medium, such as aqueous or buffer solution with detergent, normally, in an appropriate vessel.

Alternatively, 7-(carboxycarbonyI)-4-hydroxy-5-(sulfooxy)-9H-thieno[3,2-f]thiochromene-1,8-dicarboxylic acid and/or its tautomer 7-(carboxycarbonyI)-4-hydroxy-5-(sulfooxy)-7H-thieno[3,2-f]thiochromene-1,8-dicarboxylic acid may be lyophilized in the kit.

In addition to the above components, the claimed kits may further include instructions for implementing the claimed methods. These instructions may be present in the claimed kits in various forms (for example, in hard or soft copy in the form of a text and/or graphic file) in an amount of one or more copy.

### Bioluminescent Compositions

Bioluminescent compositions for use in the above applications are also provided in accordance with this invention.

In some embodiments, the compositions typically include 4-hydroxy-5-(sulfooxy)-7H-thieno[3,2-f]thiochromene-1,7,8-tricarboxylic acid and/or its tautomer 4-hydroxy-5-(sulfooxy)-9H-thieno[3,2-f]thiochromene-1,7,8-tricarboxylic acid, preferably with a buffer solution for dissolving the specified substrate and/or adding it to biological samples. 4-hydroxy-5-(sulfooxy)-7H-thieno[3,2-f]thiochromene-1,7,8-tricarboxylic acid and/or its tautomer may be dissolved in an appropriate storage medium, such as aqueous or buffer solution with detergent. Alternatively, 4-hydroxy-5-(sulfooxy)-7H-thieno[3,2-f]thiochromene-1,7,8-tricarboxylic acid and/or its tautomer may be lyophilized in the composition.

In some embodiments, the compositions typically include 7-(carboxycarbonyl)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid, preferably with a buffer solution for dissolving the specified compound and/or adding it to biological samples. 7-(carboxycarbonyI)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid may be dissolved in an appropriate storage medium, such as aqueous or buffer solution with detergent. Alternatively, 7-(carboxycarbonyI)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid may be lyophilized in the composition.

In addition to the above components, the claimed compositions may further comprise auxiliary substances, in particular adjuvants, solvents and/or fillers, such that are compatible with the compounds constituting the essence of this invention and that do not deteriorate the biological activity of these compounds.

The following examples are proposed as illustrative but not limiting.

### EXAMPLES

### Pre-Luciferin Extraction Procedure

Rinsing the lyophilized Odontosyllis undecimdonta worms with methanol and then extracting the pre-luciferin with 70% acetone. For TLC, apply the extract onto silica gel with MeOH: CH₂Cl₂ (2:1) or EtOAc: acetone: MeOH: H₂O (5:2:2:1). Collecting pink or purple samples, lyophilizing and storing at −20° C.

### Analysis of the Results Of Reversed-Phase HPLC of Lyophilized Substrate of O. Undecimdonta Biomass Fractions

Each tube containing the preserved sample was washed twice with 1 ml of water to dissolve the powder, then the solutions were lyophilized. For HPLC analysis, the samples were dissolved in 300 μl of 0.1% aqueous trifluoroacetic acid solution. A column (5 μm, 9.2×150 mm) ZORBAX SB-C18 (Agilent Technologies, USA) in Nexera X2 system (Shimadzu, Japan) was eluted with solvent A (0.1% aqueous solution of trifluoroacetic acid) and solvent B (acetonitrile) in a linear gradient from 10 to 35% (15 min) of solvent B at a flow rate of 3 ml/min. Absorption control was carried out at a wavelength of 220, 250, 330 and 410 nm (pre-luciferin absorption spectrum, FIG. 2). Fractions with bioluminescent activity were collected separately and lyophilized.

### Extraction, Separation and Purification of Luciferin and Oxyluciferin

### Water Extraction of Native Luciferase, Luciferin and Oxyluciferin from Lyophilized Odontosyllis Undecimdonta Worms

Adding 5 ml of phosphate buffer (5 mM sodium phosphate buffer, pH 7.4) to 200 mg of lyophilized worms. Pipetting the mixture into liquid nitrogen to create small drops of frozen material, then grounding them in a mortar. Adding the frozen powder to 15 ml of phosphate buffer (5 mM sodium phosphate buffer, pH 7.4) and incubating for 40 minutes in an ice bath while stirring regularly. Centrifuging the mixture at 40,000 g (4° C.) for 40 min. Collecting the supernatant comprising luciferase, luciferin and oxyluciferin, freezing the cell-free extract and storing at −70° C.

### Ion Exchange Chromatography of Aqueous Extract

Applying an aqueous extract of Odontosyllis undecimdonta to a HiTrap Fast Flow column (1.6×2.5 cm) with diethylethanolamine-sepharose (DEAE-sepharose manufactured by GE Healthcare, Uppsala, Sweden) in an Akta Prime chromatographic system (GE, USA), equilibrating and rinsing 5 mM sodium phosphate buffer (pH 7.4) at a flow rate of 5 ml/min. linear gradient eluting the column with NaCl concentration of 0 to 0.4 M (80 ml) and collecting 5 ml fractions. In order to prevent bioluminescent reaction, keeping the solvent, fractions and the column at 4° C. Analyzing in the Akta Prime chromatographic system (GE, USA). At this step, the specific activity of luciferase and luciferin was isolated and detected by pairwise mixing of all fractions. A custom-made Oberon-K luminometer (Krasnoyarsk, Russia) was used to monitor bioluminescence. At this step, luciferin and oxyluciferin (having a visible green luminescence) were not separated. All factions were frozen.

### Concentration on Strata C18 Solid Phase Extraction Cartridge

Fractions comprised both luciferin and oxyluciferin were acidified by adding trifluoroacetic acid (0.05%). Then, they were applied to a 3 ml Strata C18 solid phase extraction cartridge (Phenomenex, Torrance, USA), which had been pre-equilibrated with 0.05% aqueous trifluoroacetic acid solution. After washing with 0.05% trifluoroacetic acid, the cartridge was eluted with acetonitrile comprising 0.05% trifluoroacetic acid. At the same time, the bioluminescent activity was monitored (see below the bioluminescent activity analysis report), and the fractions were lyophilized.

### Reverse Phase Chromatography

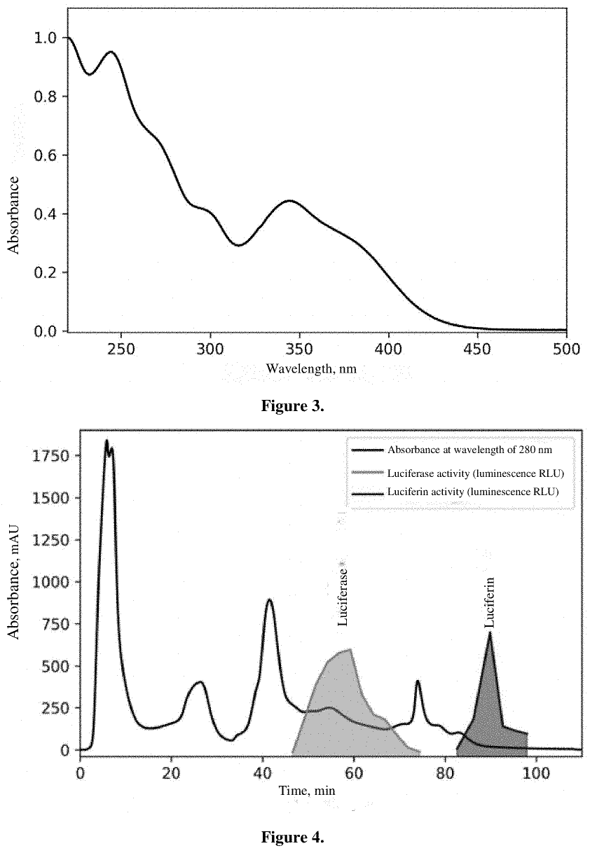

Further separating luciferin and oxyluciferin using a TSK ODS 120T 5 μm column (4.6 mm×250 mm, manufactured by LKB-Producter AB, Bromma, Sweden) in the Shimadzu chromatographic system (Shimadzu Corporation, Kyoto, Japan). Solvent A is 0.1% aqueous solution of trifluoroacetic acid, and solvent B is 0.08% solution of trifluoroacetic acid in acetonitrile, treated with 10-50% gradient B for 20 minutes at a flow rate of 1 ml/min. Carrying out absorption control at a wavelength of 220, 250, 330 and 410 nm (luciferin absorption spectrum, FIG. 3). Fractions comprising luciferin and oxyluciferin were collected separately and lyophilized.

### Luminescence Analysis

A custom-made Oberon-K luminometer (Krasnoyarsk, Russia) was used to monitor reactions. 100 μl of the reaction mixture (10 mM sodium phosphate buffer, 150 mM NaCl, 2 μl of luciferase fraction, pH 7.4) were used for each measurement. Before adding luciferin, the measurements were adjusted in accordance with the luciferase background luminescence based on the reactions monitoring for 20 s.

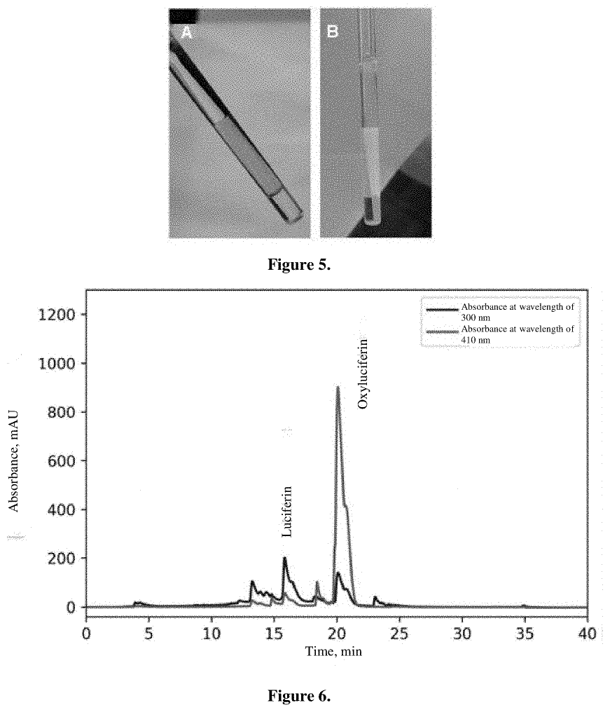

Use of ion-exchange chromatography of a cooled aqueous extract of O. undecimdonta worms allowed at the first stage to separate the substrate and enzyme-containing fractions (FIG. 4). None of the fractions in itself had bioluminescent activity. As a result of pairwise combination of all fractions the fractions comprising luciferin and luciferase were identified. The fractions comprising luciferin had a visible yellow-green color (FIG. 5A) and UV fluoresced (FIG. 5B). According to the present inventors, the observed yellow-green color of the substrate-containing fraction is most likely due to presence of oxyluciferin. This, in turn, indicated the need for further purification (FIG. 6). Fractions comprising luciferin or oxyluciferin (FIG. 6) were identified by bioluminescence analysis or absorption spectrum at 410 nm wavelength. Then, they were separated, lyophilized and used for further analysis.

### Conversion of Luciferin into Oxyluciferin, Experiment with Luminescence Reaction in Vitro

Tris-buffered saline (150 mM Tris-HCl, 1 M NaCl, pH 7.5) was used for in vitro bioluminescence reaction. Each tube contained 60 μL of pure luciferin fraction, 40 μL of tris-buffered saline and 20 μL of highly purified luciferase (see section below) or tris-buffered saline in case of negative control. Radiation was observed in the course of the reaction, while there was no luminescence in the control tube. The reaction was monitored by using 5 μl aliquots of the reaction and control mixtures, followed by stopping the reaction in 0, 5, 15, 30, 60, 120, 180, and 300 min by adding 20 μl of methanol and centrifugation, after that, the reversed-phase HPLC was used to analyze the supernatants. A Shim-Pack XR-ODS column (3.0×75 mm, 2.2 μm) was used in combination with a Nexera X2 system (Shimadzu, Japan). Solvent A is a 0.1% formic acid solution, pH 4.9, solvent B is acetonitrile. Elution was carried out in a linear gradient with the solvent B concentration from 1 to 20% (15 min) at a flow rate of 0.7 ml/min.

### Obtaining Native Luciferase and Recombinant Luciferase Samples

### Purification of Native Luciferase and Recombinant Luciferase Samples

Luciferase samples for measuring luminescence activity and in vitro conversion experiments were obtained according to the following procedure.

Purified luciferase samples were obtained as a result of sequential purification of an aqueous extract of lyophilized worms: ion-exchange chromatography in a HiTrap Fast Flow column with DEAE-Sepharose (GE Healthcare, Uppsala, Sweden), ultrafiltration in an Amicon® Ultra centrifugal filter (Merck Millipore, Germany) and gel filtration chromatography in a Superdex 200 column (Phenomenex, USA).

Recombinant luciferase was obtained from luciferase complementary DNA synthesized as a linear double-stranded DNA fragment (Twist Biosciences, USA). Cloning was performed using the Golden Gate method. Eukaryotic cell expression plasmids were assembled into the plasmid of the MoClo pICH47742 kit as a backbone, and the following parts were cloned in level 0 vectors: cytomegalovirus promoter, luciferase candidate gene, termination codon comprising a part of DNA, and SV40 polyadenylation terminator. HEK293NT cells were transfected with FuGene 6 reagent plasmid (Promega, Fitchburg, Wis., USA) according to the manufacturer's protocol. The transfected cells were grown under standard conditions.

### NMR Spectroscopy and Mass Spectrometry

### NMR Spectroscopy

The structures of luciferin, oxyluciferin, and pre-luciferin were confirmed by a set of NMR spectroscopic data (see Table 1).

¹H NMR spectra of luciferin and pre-luciferin demonstrated similar proton patterns, however, in ¹³C NMR spectrum of preluciferin a quaternary carbon signal at 177.28 ppm was observed corresponding to the carbonyl group (see Table 1).

TABLE 1
Data of NMR spectroscopy of luciferin, oxyluciferin and pre-luciferin

| | Luciferin 10° C. | | Oxyluciferin 15° C. | | Pre-luciferin 7H | | Pre-luciferin 9H | |
|---|---|---|---|---|---|---|---|---|
| | d₄⁻MeOD | | H₂O + D₂O pH 3.3 | | 15° C. D₂O pH 5.2 | | 15° C. D₂O pH 5.2 | |
| Atom^{a} | δ¹H | δ ¹³C | δ ¹H | δ ¹³C | δ ¹H | δ ¹³C | δ ¹H | δ ¹³C |
| 1 | —^{b} | 128.57 | — | 132.48 | — | 127.92 | — | 130.25 |
| 1-COOH | — | 166.10 | — | n.o. | — | n.o. | — | 175.26 |
| 2 | 8.389 | 136.77 | 8.202 | 134.76 | 7.671 | 127.80 | 7.631 | 126.90 |
| | (1H) | | (1H) | | (1H) | | (1H) | |
| 3a | — | 129.65 | — | 133.92 | — | 136.08 | — | 136.76 |
| 4 | — | n.o.^{since} | — | n.o. | — | n.o. | — | n.o. |
| 5 | — | n.o. | — | n.o. | — | n.o. | — | n.o. |
| 5a | — | 130.42 | — | 138.73 | — | 125.82 | — | 120.68 |
| 7 | 4.713 | 37.24 | — | 187.17 | 4.497 | 40.83 | — | 155.36 |
| | (1H) | | | | (1H) | | | |
| 7-COOH | — | n.o. | — | — | — | n.o. | — | n.o. |
| 7-CO | — | — | — | — | — | 177.28 | — | n.o. |
| 8 | — | n.o. | — | n.o. | — | 127.75 | — | 116.52 |
| 8-COOH | — | 168.01 | — | 168.14 | — | 172.76 | — | 171.16 |
| 9 | 8.638 | 136.87 | 9.622 | 147.92 | 8.014 | 131.15 | 4.072 | 23.00 |
| | (1H) | | (1H) | | (1H) | | (2H) | |
| 9a | — | n.o. | — | n.o. | — | n.o. | — | 119.93 |
| 9b | — | 134.10 | — | 135.56 | — | 133.77 | — | 133.94 |

Note:
^{a}The numbering of heavy atoms is shown in FIG. 7;
^{b}“—” - not applicable;
c) “n.o.” - not observed.

### Mass Spectrometry

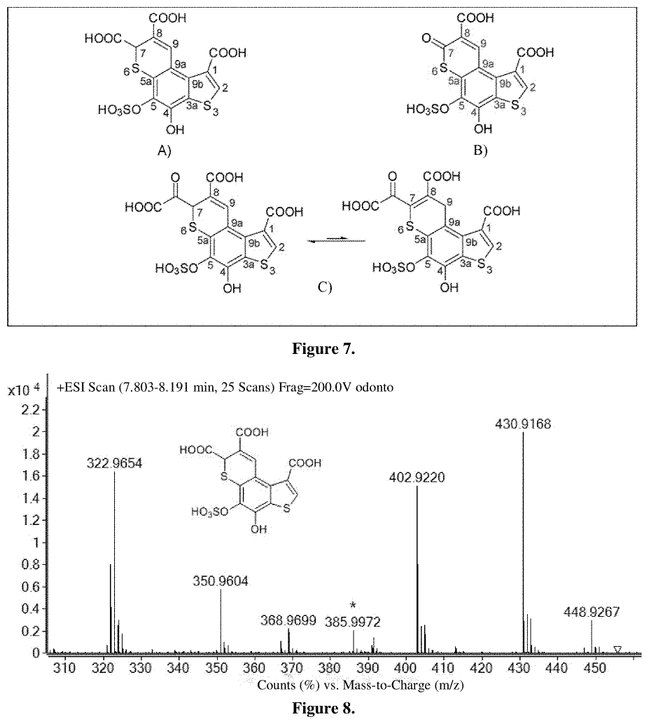

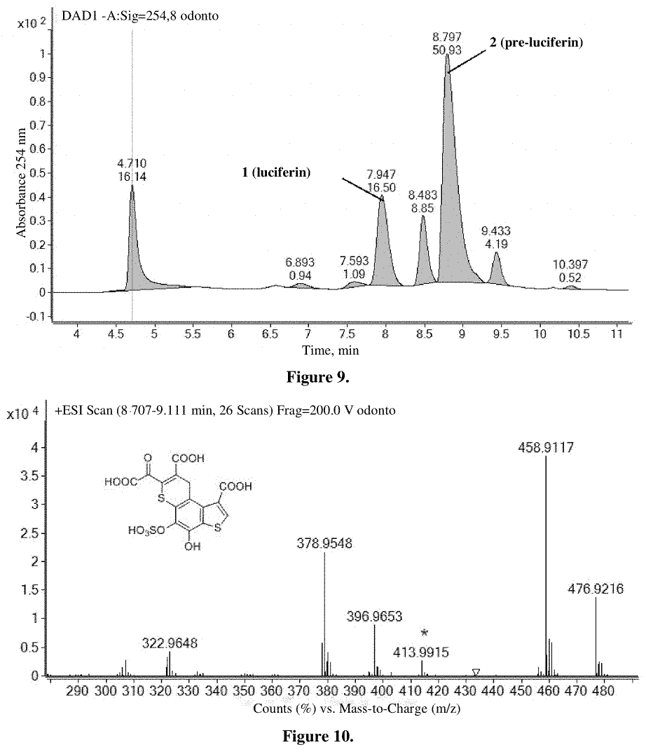

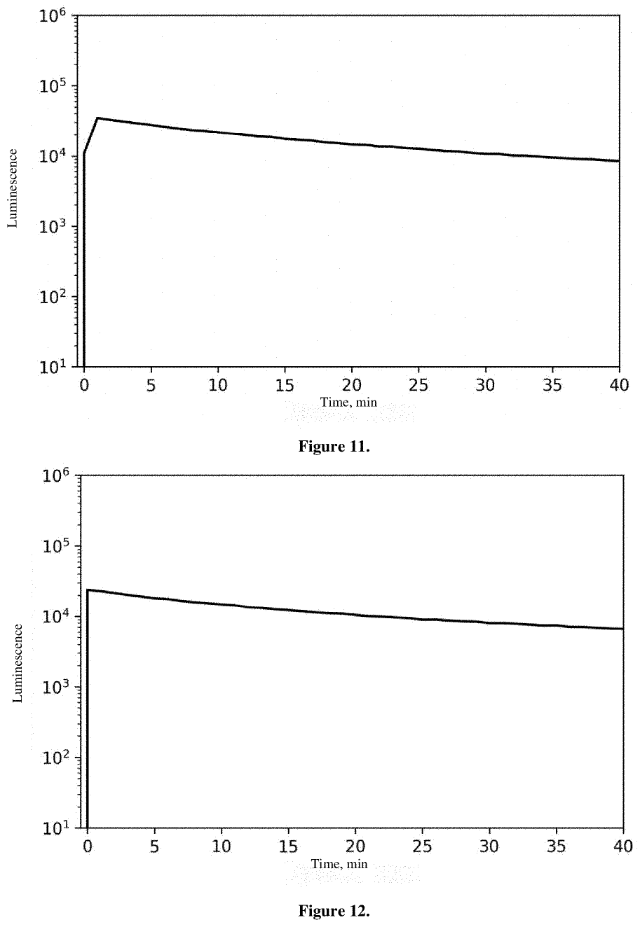

Luciferin HRMS spectrum detected the presence of a molecular ion with a mass/charge (m/z) ratio of 448.9267, corresponding to the gross formula C₁₄H₉O₁₁S₃₊ (calculated m/z value is 448.9301). It enabled to identify unambiguously the isolated luciferin as 4-hydroxy-5-(sulfooxy)-7H-thieno[3,2-f]thiochromen-1,7,8-tricarboxylic acid, which structure is shown in FIG. 8. HRMS profile of the luciferin-containing fraction main peak (FIG. 9) detected the presence of a molecular ion with a mass/charge (m/z) ratio of 476.9216, which fragmentation pattern was similar to that of luciferin (FIG. 10). The present inventors has determined that the observed ion corresponds to the molecular formula C₁₅H₉O₁₂S₃₊ (calculated m/z value is 476.9251), that enabled to unambiguously detect the presence of a carbonyl group in the composition of pre-luciferin and to identify the isolated pre-luciferin as 7-(carboxycarbonyl)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid. Based on the totality of NMR spectroscopy and mass spectrometry data, the nature of luciferin production from pre-luciferin was established as oxidative decarboxylation, and the nature of Odontosyllis undecimdonta worm bioluminescent system enzyme, catalyzing the reaction, as decarboxylase.

### Use of 4-hydroxy-5-(sulfooxy)-7H-thieno[3,2-f]thiochromene-1,7,8-tricarboxylic Acid for Detecting Luciferase in Biological Samples

Luciferin (4-hydroxy-5-(sulfooxy)-7H-thieno[3,2-f]thiochromene-1,7,8-tricarboxylic acid) was obtained as described above in the section “Extraction, separation and purification of luciferin and oxyluciferin”. Lysates of mammalian cells comprising recombinant luciferase Odontosyllis undecimdonta were used as biological samples. Cell lysates were obtained as described above in the section “Obtaining native luciferase and recombinant luciferase samples”.

1 μg of 4-hydroxy-5-(sulfooxy)-7H-thieno[3,2-f]thiochromene-1,7,8-tricarboxylic acid is dissolved in 100 μl of tris-buffered saline (150 mM tris-HCl, 1 M NaCl, pH 7.5).

During the experiment, in each case, the cold extract background luminescence was first measured, and then aliquots of a solution of 4-hydroxy-5-(sulfooxy)-7H-thieno[3,2-f]thiochromene-1,7,8-tricarboxylic acid were added.

In all cases the luminescence of biological samples was detected (FIG. 11).

### Use of 4-hydroxy-5-(sulfooxy)-7H-thieno[3,2-f]thiochromene-1,7,8-tricarboxylic Acid for Detecting Bioluminescence in Biological Samples

Luciferin (4-hydroxy-5-(sulfooxy)-7H-thieno[3,2-f]thiochromene-1,7,8-tricarboxylic acid) was obtained as described above in the section “Extraction, separation and purification of luciferin and oxyluciferin”. Extracts of Odontosyllis undecimdonta worms were used as biological samples. Cold extracts were obtained as described above in the section “Water extraction of native luciferase, luciferin and oxyluciferin from lyophilized Odontosyllis undecimdonta worms”.

1 μg of 4-hydroxy-5-(sulfooxy)-7H-thieno[3,2-f]thiochromene-1,7,8-tricarboxylic acid is dissolved in 100 μl of tris-buffered saline (150 mM tris-HCl, 1 M NaCl, pH 7.5).

During the experiment, in each case, the cold extract background luminescence was first measured, and then aliquots of a solution of 4-hydroxy-5-(sulfooxy)-7H-thieno[3,2-f]thiochromene-1,7,8-tricarboxylic acid were added.

In all cases the luminescence of biological samples was detected (FIG. 12).

### Use of pre-luciferin 7-(carboxycarbonyl)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid for Detecting Bioluminescence in Biological Samples

Pre-luciferin 7-(carboxycarbonyl)-4-hydroxy-5-(sulfooxy)-9H-thieno[3,2-f]thiochromene-1,8-dicarboxylic acid and/or its tautomer 7-(carboxycarbonyI)-4-hydroxy-5-(sulfooxy)-7H-thieno[3,2-f]thiochromene-1,8-dicarboxylic acid was obtained as described above in the section “Preluciferin extraction procedure”. Extracts of Odontosyllis undecimdonta worms were used as biological samples. Cold extracts were obtained as described above in the section “Water extraction of native luciferase, luciferin and oxyluciferin from lyophilized Odontosyllis undecimdonta worms”.

1 μg of 7-(carboxycarbonyl)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid is dissolved in 100 μl of tris-buffered saline (150 mM tris-HCl, 1 M NaCl, pH 7.5).

During the experiment, in each case, the cold extract background luminescence was first measured, and then aliquots of a solution of 7-(carboxycarbonyI)-4-hydroxy-5-(sulfooxy)-9H-thieno[3,2-f]thiochromene-1,8-dicarboxylic acid and/or its tautomer 7-(carboxycarbonyl)-4-hydroxy-5-(sulfooxy)-7H-thieno[3,2-f]thiochromene-1,8-dicarboxylic acid were added.

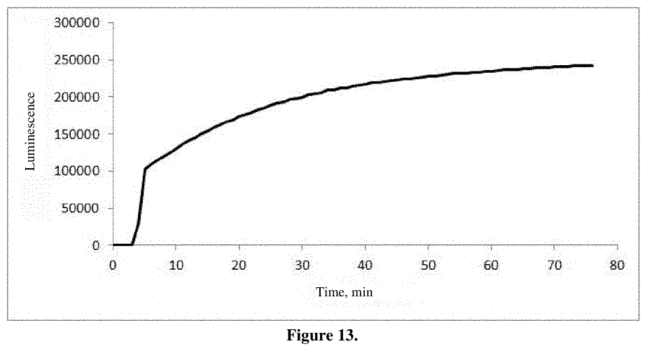

In all cases the luminescence of biological samples was detected, a typical result of luminescence detection is shown in FIG. 13.

Despite the fact that the invention is described with reference to the disclosed embodiments it should be obvious to those skilled in the art that the particular specified cases are given only for illustration purposes and they should not be considered as limiting the scope of the invention in any way. Those skilled in the art would appreciate that it is possible to implement different modifications without departing from the spirit and scope of the present invention.

1. The compound 4-hydroxy-5-(sulfooxy)-7H-thieno[3,2-f]thiochromen-1,7,8-tricarboxylic acid, characterized by the following structural formula: 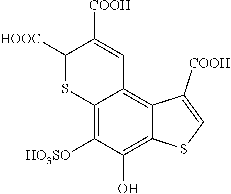
and/or its tautomer 4-hydroxy-5-(sulfooxy)-9H-thieno[3,2-f]thiochromen-1,7,8-tricarboxylic acid, characterized by the following structural formula:

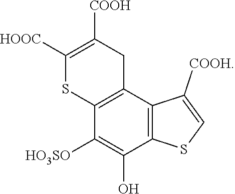

2. (canceled)

3. The compound 7-(carboxycarbonyl)-4-hydroxy-5-(sulfooxy)-9H-thieno[3,2-f]thiochromene-1,8-dicarboxylic acid, characterized by the following structural formula: 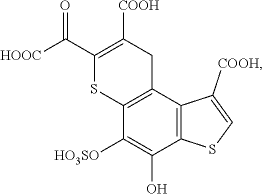
and/or its tautomer 7-(carboxycarbonyl)-4-hydroxy-5-(sulfooxy)-7H-thieno[3,2-f]thiochromene-1,8-dicarboxylic acid, characterized by the following structural formula:

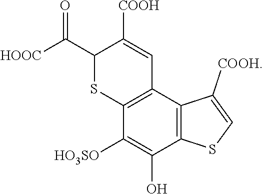

4. (canceled)

5. A kit for detecting luciferase in a biological sample comprising the compound according to claim 1.

6. A kit according to claim 5 further comprising a buffer, detergent, reducing agent and/or glycerol.

7. (canceled)

8. A bioluminescent composition, comprising the compound according to claim 1, and luciferase.

9. A composition according to claim 8, wherein a luciferase is recombinant.

10. A composition according to claim 8, wherein a luciferase is Odontosyllis undecimdonta worm luciferase.

11. A method for detecting luciferase in a biological sample comprising a luciferase and the compound according to claim 1.

12. A method according to claim 11, wherein a luciferase is Odontosyllis undecimdonta worm luciferase.

13. A method according to claim 11, wherein a luciferase is recombinant.

14. A method according to claim 11, wherein a biological sample is a tissue and/or cell.

15. A method according to claim 11, wherein a biological sample is characterized by pH within the range from 7 to 8.

16. A method according to claim 11, further comprising:
a) adding the compound to a biological sample to obtain a reaction mixture;
b) incubating the reaction mixture in conditions suitable for bioluminescence;
c) detecting bioluminescence in the reaction mixture.

17. A method according to claim 16, wherein the concentration of the compound is 0.03-300 μM.

18. A method for detecting bioluminescence in a biological sample comprising a luciferase and the compound according to claim 1.

19. A method according to claim 18, wherein a luciferase is recombinant.

20. A method according to claim 18, wherein a luciferase is Odontosyllis undecimdonta worm luciferase.

21. A method according to claim 18, wherein a biological sample is a tissue and/or cell.

22. A method according to claim 18, wherein a biological sample is characterized by pH within the range from 7 to 8.

23. A method according to claim 18, further comprising:
a) expressing luciferase gene in a biological sample;
b) adding the compound to a biological sample;
c) detecting bioluminescence.

24. A method according to claim 23, wherein the concentration of the compound is 0.03-300 μM.

25. A kit for detecting luciferase in a biological sample comprising the compound according to claim 3.

26. A kit according to claim 25, further comprising a buffer, detergent, reducing agent and/or glycerol.

27. A bioluminescent composition, comprising the compound according to claim 3, and luciferase.

28. A composition according to claim 27, wherein a luciferase is recombinant.

29. A composition according to claim 27, wherein a luciferase is Odontosyllis undecimdonta worm luciferase.

30. A method for detecting luciferase in a biological sample comprising a luciferase and the compound according to claim 3.

31. A method according to claim 30, wherein a luciferase is Odontosyllis undecimdonta worm luciferase.

32. A method according to claim 30, wherein a luciferase is recombinant.

33. A method according to claim 30, wherein a biological sample is a tissue and/or cell.

34. A method according to claim 30, wherein a biological sample is characterized by pH within the range from 7 to 8.

35. A method according to claim 30, further comprising:
a) adding the compound to a biological sample to obtain a reaction mixture;
b) incubating the reaction mixture in conditions suitable for bioluminescence;
c) detecting bioluminescence in the reaction mixture.

36. A method according to claim 35, wherein the concentration of the compound is 0.03-300 μM.

37. A method for detecting bioluminescence in a biological sample comprising a luciferase and the compound according to claim 3.

38. A method according to claim 37, wherein a luciferase is recombinant.

39. A method according to claim 37, wherein a luciferase is Odontosyllis undecimdonta worm luciferase.

40. A method according to claim 37, wherein a biological sample is a tissue and/or cell.

41. A method according to claim 37, wherein a biological sample is characterized by pH within the range from 7 to 8.

42. A method according to claim 37, further comprising:
a) expressing luciferase gene in a biological sample;
b) adding the compound to a biological sample;
c) detecting bioluminescence.

43. A method according to claim 42, wherein the concentration of the compound is 0.03-300 μM.
